(19) United States
(12) Patent Application Publication
(10) Pub. No.: US 2015/0250494 A1
(43) Pub. Date: Sep. 10, 2015

(54) Ultrasonic Follicle Unit Extraction Device and Method

(71) Applicant: Sanusi UMAR, Redondo Beach, CA (US)

(72) Inventor: Sanusi Umar, Redondo Beach, CA (US)

(21) Appl. No.: 14/388,081

(22) PCT Filed: Nov. 26, 2013
(86) PCT No.: PCT/US13/72090

§ 371 Date: Sep. 25, 2014

Related U.S. Application Data
(63) Continuation-in-part of application No. 14/090,968, filed on Nov. 26, 2013, now abandoned.
(60) Provisional application No. 61/729,733, filed on Nov. 26, 2012.
(60) Provisional application No. 61/874,664, filed on Sep. 6, 2013.

(51) Int. Cl. A61B 17/3205; A61B 17/32

(52) U.S. Cl. CPC A61B 17/32053; A61B 17/320068; A61B 2017/00752

(57) ABSTRACT

A follicle unit extraction system includes a generally tubular follicle punch having a distal cutting edge, and an ultrasonic transducer coupled to the punch to impart rapid, incremental, distally-directed cutting force pulses to the punch during the cutting operation. The punch is being mounted at the forward end of a handpiece, and the ultrasonic transducer is resiliently mounted within the handpiece and releasably coupled to the punch for imparting ultrasonic vibration to the punch.

### BACKGROUND

Follicular Unit Extraction (“FUE”) is a surgical hair transplantation technique that involves moving skin containing hair follicles from one part of the body (the donor site) to bald or balding parts (the recipient site). Hair naturally grows in follicles that contain groupings of 1 to 4 hairs, and the transplant technique typically moves the 1-4 hair “follicular units” from the donor site to the recipient site.

The follicle units (sometimes referred to as “grafts”) are typically removed from the donor site using punches of between 0.7 mm and 1.25 mm in diameter. The punches are typically tubular bodies having a skin-contacting cutting edge, and are typically mounted in a hand-held electric-powered tool that causes the punch to rotate or oscillate as the cutting device is brought into contact with the donor site, but are sometimes used manually.

### SUMMARY OF DESCRIPTION

An FUE device constructed in accordance with the invention comprises a follicular unit extraction device comprising a handpiece, a generally tubular follicle punch mounted at a forward end of the handpiece, and an ultrasonic transducer resiliently mounted within the handpiece and releasably coupled to the punch for imparting ultrasonic vibration to the punch. The use of the ultrasonic movement permits the surgeon or other operator of the equipment to better use his/her “fine motor” muscle movement to more precisely make the required incisions with greater sensitivity and finesse.

Preferably, the handpiece carries a liquid delivery system that includes a passageway configured to conduct liquid from an external source to the cutting edge of the punch during cutting and extraction of the follicle. Preferably, the liquid passageway includes at least a segment lying within the handpiece's interior, and the liquid is conducted to the cutting edge by being introduced into the punch's lumen at its proximal end. The conducted liquid can thereby provide a source of cooling for the transducer and punch while also lubricating and cushioning the follicle during the cutting and extraction process.

Any of a number of liquids can be used, including physiologic or other solutions, and may also be a fluid that prolongs or sustains graft or tissue viability.

A flow-rate controller such as an adjustable pump or adjustable flow restrictor is preferably utilized to control the flow rate of the liquid within the liquid passageway and punch to produce a desired drip rate during the cutting and extraction procedure, and to selectively increase the flow rate and consequential pressure of the liquid flowing through the punch to clear impacted follicles and/or debris from the punch. The flow-rate controller can be conveniently located between the liquid source and the handpiece, although other locations can be utilized without departing from the scope of the invention.

Preferably, the handpiece is operable to rotate the punch, rotationally pivot the punch, and/or reciprocally pivot the punch, with or without ultrasonic vibratory movement during the cutting operation. Likewise, the preferred FUE device is selectively operable to generate ultrasonic vibratory movement with or without the pivoting movement during the cutting operation.

The transducer can be either the piezo-electric or the magnetostrictive type, similar to those used in dental scalers. Frequencies of 25-35 kHz appears acceptable, but frequencies above and below the foregoing range are within the scope of the invention. The piezo-electric transducer is currently preferred owing to its production of substantially less heat during operation, as well as its ability to operate at a higher ultrasonic frequency than a magnetostrictive transducer. The liquid path within the handpiece can be configured to draw heat from the transducer; a thin wall preferably separates the transducer from the liquid path to preserve the sterility of the liquid prior to its application to the donor site.

In practice, it has been found that an adjustable degree of longitudinally reciprocating ultrasonic movement is desirable in that the appropriate degree of movement is a function of the subject's skin thickness and tissue, with higher settings being suitable when cutting through thicker skin or scar tissue for example.

### DETAILED DESCRIPTION OF PREFERRED EMBODIMENT

An FUE device constructed in accordance with the invention comprises a follicular unit extraction device comprising a handpiece, a generally tubular follicle punch mounted at a forward end of the handpiece, and an ultrasonic transducer resiliently mounted within the handpiece and releasably coupled to the punch for imparting ultrasonic vibration to the punch.

As a step towards developing the invention, a follicle punch having a distal cutting edge has been placed in an ultrasonic dental scaler handpiece in lieu of the scaler tip after having been suitably machined at is proximal end to be secured to the handpiece in the manner by which the scaler tip was secured.

Liquid from a source external to the handpiece was introduced into the proximal end of the follicle punch, also referred to as the lumen utilizing the dental scaler handpiece's fluid path that would have normally introduced liquid into the scaler tip.

An adjustable pump controlled the flow rate of the liquid to the handpiece and punch to produce a desired drip rate during the follicle cutting procedure, and was used to increased the flow rate, and consequential pressure of the liquid flowing through the punch, to clear impacted follicles and/or debris from the punch.

The ultrasonic dental scaler included means for adjusting the amplitude of the ultrasonic pulses, and was used to provide appropriate pulse strengths to the follicle punch during the cutting operation.

Although the present invention and its advantages have been described in detail, it should be understood that various changes, substitutions and alterations can be made herein without departing from the spirit and scope of the invention as will be defined by appended claims.

I claim:

1. A follicle unit extraction system comprising
a generally tubular follicle punch having a distal cutting edge, and
an ultrasonic transducer coupled to the punch to impart rapid, incremental, distally-directed cutting force pulses to the punch during the cutting operation.

2. The follicle unit extraction system of claim 1 including a handpiece, the follicle being mounted at a forward end of the handpiece, and the ultrasonic transducer being resiliently mounted within the handpiece and releasably coupled to the punch for imparting ultrasonic vibration to the punch.

3. The follicle unit extraction system of claim 2 wherein the handpiece includes an internal liquid passageway for conducting liquid from an external source into the interior of the punch at the punch's proximal end.

4. The follicle unit extraction system of claim 3 including
a flow-rate controller for controlling the flow rate of the liquid being conducted within the liquid passageway and punch to produce a desired drip rate during the cutting and extraction procedure, and for selectively increasing the flow rate and consequential pressure of the liquid flowing through the punch to clear impacted follicles and/or debris from the punch.

4. A method for cutting a follicle unit at a donor site prior to extraction comprising the step of applying ultrasonic vibratory cutting force against the tissue at the donor site during the cutting operation.

5. A method for cutting a follicle unit at a donor site prior to extraction comprising the steps of:
bringing a generally tubular follicle punch having a distal cutting end region into contact with the skin at a donor site;
inserting the punch distally into the skin in such a way that the follicle enters the punch's interior as the skin and underlying tissue circumscribing the follicle are cut; and
applying ultrasonic, incremental, distally-directed cutting force pulses to the tissue via the punch during the cutting process.
